(19) United States
(12) Patent Application Publication
(10) Pub. No.: US 2025/0000581 A1
(43) Pub. Date: Jan. 2, 2025

(54) COMBINED LASER ATHERECTOMY AND PRESSURE WAVE DEVICE

(71) Applicant: KONINKLIJKE PHILIPS N.V., EINDHOVEN (NL)

(72) Inventors: James David CEZO, COLORADO SPRINGS, CO (US); Bernardus Hendrikus Wilhelmus HENDRIKS, EINDHOVEN (NL); Jarich Willem SPLIETHOFF, UTRECHT (NL); Marco LAI, EINDHOVEN (NL); Gerhardus Wilhelmus LUCASSEN, EINDHOVEN (NL); Joanneke Gerrigje GROEN, VELDHOVEN (NL); Charles Frederik SIO, EINDHOVEN (NL)

(21) Appl. No.: 18/755,936

(22) Filed: Jun. 27, 2024

Related U.S. Application Data
(60) Provisional application No. 63/523,697, filed on Jun. 28, 2023.

(51) Int. Cl. A61B 18/26; A61B 18/00; A61B 18/20; A61B 18/22; A61B 90/00

(52) U.S. Cl. CPC A61B 18/26; A61B 90/39; A61B 2018/00404; A61B 2018/207; A61B 2018/2211; A61B 2018/2266; A61B 2018/263; A61B 2090/3966; A61B 2218/002; A61B 2218/007

(57) ABSTRACT

An intraluminal device for delivering laser light and pressure waves includes a flexible elongate member positionable within a body lumen. The member includes an outer sheath; a laser catheter attached within the sheath; a sealed lens assembly located at the sheath's distal end; and a fluid chamber within the sheath, distal of the laser catheter and proximal of the lens assembly. The outer sheath includes a sealed acoustic window radially outward from the fluid chamber. When the chamber is filled with a fluid, a laser beam emitted by the laser catheter causes the photoreactive fluid to generate a pressure wave. When the chamber is filled with the fluid, a second laser beam emitted by the laser catheter exits via the transparent fluid and the sealed lens assembly. The sealed acoustic window and sealed lens assembly prevent the fluid from entering the body lumen.

### TECHNICAL FIELD

The subject matter described herein relates to systems, devices, and methods for generating a shockwave and directing ablative laser light within a body lumen of a patient. The combined laser atherectomy and pressure wave device disclosed herein has particular but not exclusive utility for intravascular lithotripsy, e.g., fracturing of calcified vascular stenoses inside of a blood vessel.

### BACKGROUND

Many individuals (especially elderly individuals) suffer from progressively enlarging vascular calcium mineral deposits. Such vascular calcification can cause symptoms such as compromised vascular integrity, vascular stenoses, hypertension, enlargement of the heart, ischemia, and congestive heart failure. The severity and extent of mineralization are strong predictors for morbidity and mortality. Vascular calcification may be recognized as a pathobiological process similar in some ways to bone formation. Vascular (e.g., arterial and venous) calcifications can be a persistent problem when treating coronary or peripheral vessels.

Vascular calcification may in many cases be co-morbid with other vascular diseases such as arterial plaques, which may be treated through combinations of laser atherectomy, balloon angioplasty, and/or stenting. However, vascular calcifications may be rigid enough to resist expansion by a stent or balloon, making such combined stenoses difficult to treat.

The information included in this Background section of the specification, including any references cited herein and any description or discussion thereof, is included for technical reference purposes only and is not to be regarded as subject matter by which the scope of the disclosure is to be bound.

### SUMMARY

Disclosed is a combined laser atherectomy and pressure wave device which has particular, but not exclusive, utility for ablating vascular plaques and fracturing calcified vascular stenoses. The combined laser atherectomy and pressure wave device has a shockwave functionality for cracking the calcium in the radial direction and a penetrating laser atherectomy device to produce a crossing through the chronic total occlusion (CTO), without requiring mechanical movement of the laser catheter with respect to the outer sheath, and without releasing a contrast agent or other photoreactive liquid into the patient's body. The combined laser atherectomy and pressure wave device includes both a laser catheter and a sealed reaction chamber or fluid chamber, in acoustical communication with the blood volume inside the vessel, where the photoreaction can take place without release of the photoreactive fluid or its reaction products into the patient's bloodstream.

One general aspect includes an intraluminal device for delivering laser light and pressure waves. The device includes a flexible elongate member configured for positioning within a body lumen and comprising: an outer sheath; a laser catheter disposed within the sheath and fixedly coupled to the outer sheath; a sealed lens disposed at a distal end of the outer sheath; and a fluid chamber disposed within the sheath, distal of a distal end of the laser catheter and proximal of the sealed lens, wherein the fluid chamber is configured to be filled with a fluid; wherein the outer sheath comprises a sealed acoustic window disposed radially outward from the fluid chamber, wherein a first beam of laser light emitted by the laser catheter is configured to cause the fluid to generate a pressure wave, wherein a second beam of laser light emitted by the laser catheter is configured to be transmitted via the fluid and the sealed lens, wherein the sealed acoustic window and sealed lens prevent the fluid from entering the body lumen.

Implementations may include one or more of the following features. In some aspects, the flexible elongate member further may include: an inflow channel disposed within the outer sheath and in fluid communication with the fluid chamber; and an outflow channel disposed within the outer sheath and in fluid communication with the fluid chamber. In some aspects, the fluid may include at least one of a photoreactive fluid or an optically transmissive fluid, where the fluid chamber is configured to be filled, via the inflow channel, with the photoreactive fluid and with the optically transmissive fluid at different times, and where the fluid chamber is configured to be emptied, via the outflow channel, of the photoreactive fluid and of the optically transmissive fluid at different times. In some aspects, the optically transmissive fluid may include at least one of water, saline, or air. In some aspects, the first beam of laser light comprises a first portion of a same beam of laser light emitted by the laser catheter and the second beam of laser light comprises a second portion of the same beam of laser light, and the first portion of the same beam of laser light is transmitted via the fluid and the sealed lens simultaneously as the second portion of the same beam of laser light causes the fluid to generate the pressure wave. In some aspects, the flexible elongate member further may include a beam splitter disposed within the outer sheath, where the beam splitter directs the first portion of the same beam in a first direction and directs the second portion of the same beam in a different, second direction. In some aspects, the first beam of laser light comprises a first wavelength, and the second beam of laser light comprises a different, second wavelength.

In some aspects, the sealed acoustic window is arranged relative to the fluid chamber such that the pressure wave is transmitted radially outward from the outer sheath into the body lumen. In some aspects, the sealed lens is arranged relative to the fluid chamber such that the second beam of laser light is transmitted, into the body lumen, in a direction distal of the distal end of the sheath. In some aspects, the laser catheter may include an ultraviolet laser catheter. In some aspects, the sealed lens may include a flat proximal surface and a flat distal surface. In some aspects, the sealed lens may include a convex proximal surface. In some aspects, the sealed lens may include a convex distal surface. In some aspects, the device further includes a relay lens assembly and the relay lens assembly includes the sealed lens. In some aspects, the flexible elongate member further may include a radiopaque marker positioned on the outer sheath proximate to the acoustic window. In some aspects, the intraluminal device may include a guidewire lumen extending longitudinally through the laser catheter and the sealed lens. Implementations of the described techniques may include hardware, a method or process, or computer software on a computer-accessible medium.

One general aspect includes an intravascular treatment system that includes a laser light source; a photoreactive fluid source with a photoreactive fluid; an optically transmissive fluid source with an optically transmissive fluid; an intravascular device configured to deliver laser light and pressure waves into a blood vessel, where the intravascular device may include: a flexible elongate member configured for positioning within the blood vessel and may include: an outer sheath; a laser catheter disposed within the sheath and fixedly coupled to the outer sheath; a sealed lens disposed at a distal end of the outer sheath; and a fluid chamber disposed within the sheath, distal of a distal end of the laser catheter and proximal of the sealed lens. The outer sheath may include a sealed acoustic window disposed radially outward from the fluid chamber. When the fluid chamber is filled with the photoreactive fluid, a first beam of laser light emitted by the laser catheter causes the photoreactive fluid to generate a pressure wave to perform lithotripsy of a blood vessel blockage. When the fluid chamber is filled with the optically transmissive fluid, a second beam of laser light emitted by the laser catheter is transmitted via the optically transmissive fluid and the sealed lens to perform laser atherectomy of the blood vessel blockage. The sealed acoustic window and sealed lens prevent the photoreactive fluid and the optically transmissive fluid from entering the blood vessel. Other embodiments of this aspect include corresponding computer systems, apparatus, and computer programs recorded on one or more computer storage devices, each configured to perform the actions of the methods.

One general aspect includes a laser atherectomy and pressure wave delivery method. The method includes, with a processor that has a memory: activating a pump; via the pump and at least one inflow channel disposed within a flexible elongate member positioned within a blood vessel, controlling filling of a fluid chamber disposed within the flexible elongate member with an optically transmissive fluid; activating a laser catheter disposed within the flexible elongate member, such that a first beam emitted by the laser catheter passes through the optically transmissive fluid and a sealed lens for laser atherectomy of a blood vessel blockage; via the pump and a second inflow channel disposed within the flexible elongate member, controlling the filling of the fluid chamber with a photoreactive fluid; and activating the laser catheter, such that a second beam emitted by the laser catheter passes through the fluid chamber, causing the photoreactive fluid to produce a pressure wave for lithotripsy of the blood vessel blockage. The sealed lens prevents the optically transmissive fluid and the photoreactive fluid from entering the blood vessel. Other embodiments of this aspect include corresponding computer systems, apparatus, and computer programs recorded on one or more computer storage devices, each configured to perform the actions of the methods.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. Other embodiments of this aspect include corresponding computer systems, apparatus, and computer programs recorded on one or more computer storage devices, each configured to perform the actions of the methods.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the combined laser atherectomy and pressure wave device, as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a schematic, diagrammatic representation of an example intravascular treatment system with combined laser atherectomy and pressure wave device, in accordance with at least one aspect of the present disclosure.

FIG. 2 is a diagrammatic, schematic, lateral cross-sectional view of at least a portion of an example combined laser atherectomy and pressure wave device, in accordance with at least one aspect of the present disclosure.

FIG. 3 is a schematic, diagrammatic, longitudinal cross-sectional view of at least a portion of an example combined laser atherectomy and pressure wave device, in accordance with at least one aspect of the present disclosure.

FIG. 4 is a schematic, diagrammatic, longitudinal cross-sectional view of at least a portion of an example combined laser atherectomy and pressure wave device, in accordance with at least one aspect of the present disclosure.

FIG. 5 is a schematic, diagrammatic, longitudinal cross-sectional view of at least a portion of an example combined laser atherectomy and pressure wave device, in accordance with at least one aspect of the present disclosure.

FIG. 6 is a schematic, diagrammatic, longitudinal cross-sectional view of at least a portion of an example combined laser atherectomy and pressure wave device, in accordance with at least one aspect of the present disclosure.

FIG. 7 is a schematic, diagrammatic, longitudinal cross-sectional view of at least a portion of an example combined laser atherectomy and pressure wave device, in accordance with at least one aspect of the present disclosure.

FIG. 8 is a schematic, diagrammatic, longitudinal cross-sectional view of at least a portion of an example combined laser atherectomy and pressure wave device, in accordance with at least one aspect of the present disclosure.

FIG. 9 is a schematic, diagrammatic, longitudinal cross-sectional view of at least a portion of an example combined laser atherectomy and pressure wave device, in accordance with at least one aspect of the present disclosure.

FIG. 10A is a schematic, diagrammatic, longitudinal cross-sectional view of at least a portion of an example combined laser atherectomy and pressure wave device, in accordance with at least one aspect of the present disclosure.

FIG. 10B is a schematic, diagrammatic, longitudinal cross-sectional view of at least a portion of an example combined laser atherectomy and pressure wave device, in accordance with at least one aspect of the present disclosure.

FIG. 11 shows a flow diagram of an example laser atherectomy and calcified stenosis fracturing method, in accordance with at least one aspect of the present disclosure.

FIG. 12 is a schematic, diagrammatic, longitudinal cross-sectional view of at least a portion of blood vessel undergoing treatment by an example combined laser atherectomy and pressure wave device, in accordance with at least one aspect of the present disclosure.

FIG. 13 is a schematic, diagrammatic, longitudinal cross-sectional view of at least a portion of blood vessel undergoing treatment by an example combined laser atherectomy and pressure wave device, in accordance with at least one aspect of the present disclosure.

FIG. 14 is a schematic, diagrammatic, longitudinal cross-sectional view of at least a portion of blood vessel undergoing treatment by an example combined laser atherectomy and pressure wave device, in accordance with at least one aspect of the present disclosure.

FIG. 15 is a schematic, diagrammatic, longitudinal cross-sectional view of at least a portion of blood vessel undergoing treatment by an example combined laser atherectomy and pressure wave device, in accordance with at least one aspect of the present disclosure.

FIG. 16 is a schematic, diagrammatic, longitudinal cross-sectional view of at least a portion of blood vessel undergoing treatment by an example combined laser atherectomy and pressure wave device, in accordance with at least one aspect of the present disclosure.

FIG. 17 is a schematic, diagrammatic, longitudinal cross-sectional view of at least a portion of blood vessel undergoing treatment by an example combined laser atherectomy and pressure wave device, in accordance with at least one aspect of the present disclosure.

FIG. 18 is a schematic, diagrammatic, longitudinal cross-sectional view of at least a portion of blood vessel undergoing treatment by an example combined laser atherectomy and pressure wave device, in accordance with at least one aspect of the present disclosure.

FIG. 19 is a schematic, diagrammatic, longitudinal cross-sectional view of at least a portion of blood vessel after treatment by a combined laser atherectomy and pressure wave device, in accordance with at least one aspect of the present disclosure.

FIG. 20 is a schematic, diagrammatic, longitudinal cross-sectional view of at least a portion of blood vessel after treatment by a combined laser atherectomy and pressure wave device, in accordance with at least one aspect of the present disclosure.

FIG. 21 is a schematic, diagrammatic, longitudinal cross-sectional view of at least a portion of blood vessel after treatment by a combined laser atherectomy and pressure wave device and a balloon catheter, in accordance with at least one aspect of the present disclosure.

FIG. 22 is a schematic diagram of a processor circuit, according to aspects of the present disclosure.

### DETAILED DESCRIPTION

The present disclosure provides a combined laser atherectomy and pressure wave device for an intravascular treatment system. The combined laser atherectomy and pressure wave device is configured both to perform laser ablation and to fracture vascular calcifications via a shockwave, and thus help to enable dilation of the stenosis. Calcifications tend to be well rooted into soft tissue, or even completely encapsulated by soft tissue, such that fracturing the calcification does not generally release the calcified material into the bloodstream. The fracturing process may for example be analogous to breaking up a bag of frozen-together ice cubes, without opening the bag.

The present disclosure may for example be applicable to treating calcified chronic total occlusions (CTOs) with a catheter device capable of producing shockwaves to modify the calcium in or surround the CTO. The presence of calcium lesions in and surrounding the total occlusion may preclude typical catheter-based treatments (e.g., inflating a balloon or placing a stent). Devices such as electrically induced shockwave balloon catheters (e.g., to modify the calcium through fracturing, a process known as lithotripsy) can be used, such that ballooning or stenting becomes possible. However, such an electrically actuated shockwave balloon catheter may only be effective when it can enter the stenosis. In the case of a calcified CTOs, this may be difficult or impossible.

As described for example in U.S. Pat. No. 11,058,492, incorporated by reference as though fully set forth herein, such calcified CTOs may be treatable with a device that is capable of penetrating a calcified and/or fibrous vascular occlusion, particularly a calcified cap(s), and disrupting at least a portion of the vascular occlusion as the device penetrates and traverses the total occlusion. The device may be able to produce laser induced pressure waves to the vascular occlusion to disrupt the calcified and/or fibrous portions. However, existing systems require a laser catheter capable of translating longitudinally within a separate sheath. The laser catheter can ablate plaque and other materials, including but not limited to atherosclerotic plaques, lipidous plaques, thrombus, neointimal hyperplasia, fibrous and calcific plaques, without damaging the vessel wall. When the laser is partially retracted within the sheath and a contrast fluid or other photoreactive fluid is introduced into the vessel via the sheath, the laser can be used to initiate an exothermic reaction in the photoreactive fluid that generates shockwaves (also known as pressure waves or acoustic shock). These shockwaves can fracture the calcified tissue embedded in the vessel wall, without damaging the soft tissue of the vessel wall itself.

In some aspects, U.S. Pat. No. 11,058,492 (see FIG. 33 therein) describes the combination of first performing an atherectomy procedure on the vascular obstruction with the laser catheter beyond the distal end of the surrounding sheath, then followed by a pressure wave or lithotripsy procedure. In this second procedure, the laser catheter and the sheath are positioned such that a pressure wave reflective element within the sheath is adjacent an occlusion with the vessel and the distal end of the laser catheter is disposed within the pressure wave reflective element and the sheath. Photoreactive liquid is introduced and the laser is activated to disrupt the obstruction. Hence, this reference discloses a laser catheter and a sheath where during the atherectomy the laser is beyond the end of the sheath and during the pressure wave procedure the laser catheter is completely inside the sheath.

The various steps for this combined procedure require various manual movements by the physician. For example, the step where the laser catheter should be retracted into the surrounding sheath at the correct location may be particularly critical. In addition, some patients may be allergic or sensitive to the photoreactive liquid or the photoreaction products. Even in cases where an allergy is not present, the photoreactive liquid or its reaction products may cause unwanted side effects (e.g., in patients with kidney failure or other vulnerabilities). Such side effect may include, but are not limited to, rapid or slow heart rate, low blood pressure, asthma attack, and complete circulatory collapse/shock.

Examples of other aspects are for instance where the atherectomy is not performed by the laser but by the sheath, for instance by incorporating a “jackhammer” at the tip of the sheath. When shockwaves are produced this will also move forward the tip of the sheath that then functions as a jackhammer as described in U.S. Pat. Nos. 11,058,492 and 11,058,492. However, this method suffers from similar problems as described above. Therefore, to further simplify the workflow steps, the need for this retracting and advancing should be eliminated.

Thus, the present disclosure provides a device that has a highly localized shockwave functionality for cracking the calcium in the radial direction and a forward-penetrating device to produce a crossing through the CTO, all without requiring mechanical movement of the laser catheter with respect to outer sheath, and without releasing a contrast agent or other photoreactive liquid into the patient's body.

In some aspects, the combined laser atherectomy and pressure wave device includes both a laser catheter and a reaction chamber or fluid chamber, in acoustical communication with the blood volume inside the vessel, where the photoreaction can take place without release of the photoreactive fluid or its reaction products into the environment surrounding the device. Depending on the implementation, the combined laser atherectomy and pressure wave device may produce a shockwave that can travel into the blood vessel, or may produce repeated shocks, or may produce a time-varying (e.g., sinusoidal) pressure. A shockwave may for example occur when the expansion of a fluid, or as a result of a photochemical reaction between a laser light and photoreactive medium.

The present disclosure provides a system consisting of a laser catheter, a sheath around the laser catheter terminated by an optical lens, and a reaction chamber or fluid chamber occupying the space between the laser catheter and the exit and lens, and fluid channels connected to the reaction chamber or fluid chamber. The liquid in the reaction chamber or fluid chamber can be switch from a transmissive liquid that does not react with the wavelength of light used during laser ablation to an absorptive, photoreactive liquid (e.g., radiopaque contrast liquid) that is able to generate shockwaves when exposed to the laser light. Furthermore, the sheath includes acoustic windows (e.g., one or more portions made from a polymer or hydrogel which has similar speed of sound to the fluid medium which allows shockwaves to pass from the interior to the exterior of the device, while the liquid remains confined inside the reaction chamber or fluid chamber. When the liquid is transmissive to laser light (e.g., saline or air for UV laser wavelengths), the system functions as a laser atherectomy device. When the liquid is absorptive of laser light (e.g. contrast agent with UV light or other photoreactive liquid) the system can produce shockwaves that can pass radially outward through the acoustic windows of the sheath.

In some aspects, a guidewire lumen is present, and is in fluid communication with the patient's bloodstream but not with the fluid channels or reaction chamber/fluid chamber. In some aspects, the lens can have optical power to minimize the divergence of the beam exiting the laser sheath to keep the light confined to the tissues in front of the catheter for the purposes of atherectomy. In some aspects, the lens system can be a flat exit window or may include one or more curved surfaces that have optical power (e.g., light-bending capability). In some aspects, the lens surfaces preferably have an anti-reflective coating, both to minimize light loss due to refractive index mismatch and to reduce the damage of the laser catheter due to the reflection of the lens surfaces. However, if the refractive index between the fluid and the lens match too well, then the fluid-lens boundary will be “invisible” and may thus not be operative to bend light rays. In some aspects, the lens surface facing the laser catheter is convex in order that reflected light diverges before reaching the laser catheter again.

The present disclosure aids substantially in the treatment of calcified stenoses, by improving a clinician's ability to deliver high pressures to the calcification without introducing chemicals into the patient's bloodstream. In acoustic communication with blood in the vessel, the combined laser atherectomy and pressure wave device disclosed herein provides a practical ability to deliver shockwaves, pressure waves, or vibrations to a stenosis, whether before, during, or after the delivery of ablative laser light for atherectomy, without the need to move the laser catheter relative to the sheath, and without introducing contrast fluid or other reactive materials into the patient's bloodstream. For example, the contrast fluid can be toxic to patients, and some patients cannot tolerate contrast fluid being introduce into their blood vessel/blood. By completely sealing the fluid inside the device, even patients that cannot tolerate contrast fluid can have lithotripsy/pressure wave treatment. This improved atherectomy technique transforms a solid calcification into a number of tissue-bound fragments, without the normally routine need to ablate or cut the calcified tissue or disturb the soft tissue around it, and without the need to move the laser catheter relative to the sheath. Indeed, the distal, energy-delivering portion of the device may be completely sealed. This unconventional approach improves the functioning of the intravascular treatment system, by permitting shockwave therapy and laser atherectomy to be delivered by a single, sealed device.

The combined laser atherectomy and pressure wave device may be controlled manually, or through an automated process at least partially viewable on a display and executing on a processor that accepts user inputs from a keyboard, mouse, touchscreen interface, or other user interface. In that regard, the control process performs certain specific operations in response to different inputs or selections made at different times. Certain structures, functions, and operations of the processor, display, sensors, and user input systems are known in the art, while others are recited herein to enable novel features or aspects of the present disclosure with particularity.

These descriptions are provided for exemplary purposes only and should not be considered to limit the scope of the combined laser atherectomy and pressure wave device. Certain features may be added, removed, or modified without departing from the spirit of the claimed subject matter.

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one aspect may be combined with the features, components, and/or steps described with respect to other aspects of the present disclosure. For the sake of brevity, however, the numerous iterations of these combinations will not be described separately.

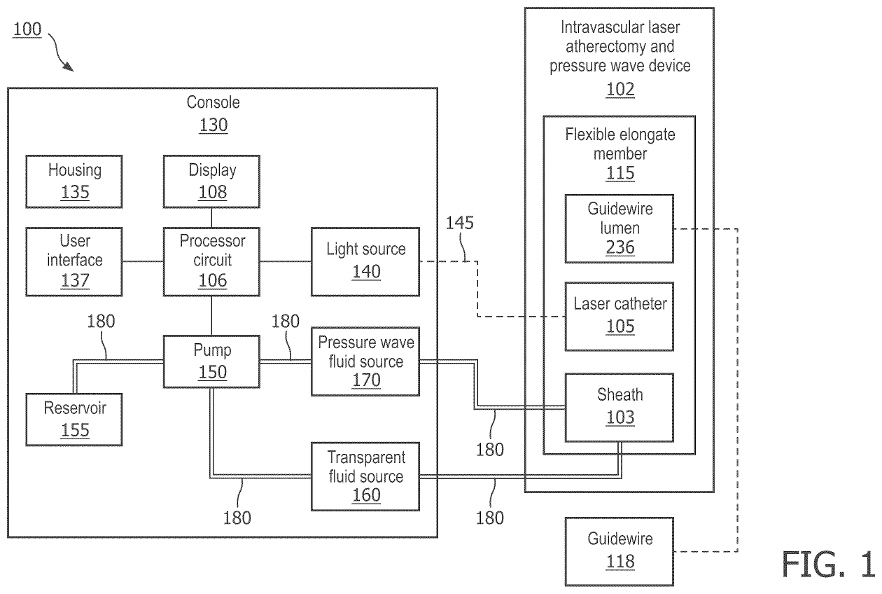

FIG. 1 is a schematic, diagrammatic representation, in block diagram form, of an example intravascular treatment system 100 with combined laser atherectomy and pressure wave device 102, in accordance with at least one aspect of the present disclosure. The intravascular treatment system 100 includes a console or workstation 130 and an intravascular combined laser atherectomy and pressure wave device 102.

The console 130 of an unsealed laser-induced pressure wave emitting catheter and sheath is described for example in U.S. Pat. Nos. 11,058,492 and 11,246,659, along with background information relating to the problems addressed by the present disclosure. In the example shown in FIG. 1, the console 130 includes a housing 135, a display 108 (e.g., a touchscreen display), a user interface 137 (which may for example include virtual controls on the touchscreen display and/or physical knobs, buttons, etc.), a processor circuit 106, and a light source 140, such as an UV laser light source.

In some aspects, the console 130 also includes a pump 150 capable of delivering photoreactive liquid from a pressure wave fluid source 170 and/or air or saline from a transparent fluid source 160, as well as a reservoir 155 for receiving effluent photoreactive liquid or transparent liquid. In other aspects, the pump 150, pressure wave fluid source 170, transparent fluid source 160, and/or reservoir 155 may be separate from the console 130. In some aspects, the pump 150 may be an electromechanical pump. In some aspects, the pump 150 may be multiple pumps (e.g., one pump for pressure wave fluid and one for transparent fluid). In some aspects, the pump 150 may be or may include a manually operated plunger.

Components shown as being inside the console 130 may be in separate housings (e.g., different consoles). For example, there could be a light source console and a fluid sources console. The light source console can be a housing with the light source, a processor circuit, display, user interface for control of the light source. The fluid sources console could be a housing with the shockwave fluid source, transparent fluid source, a processor circuit, display, user interface for control of the fluid sources. There could be separate consoles for the different fluid sources (with separate housings, processor circuits, displays, user interfaces). Still other arrangements are possible.

The console 130 interfaces with a combined laser atherectomy and pressure wave device 102 (also known as an intravascular laser atherectomy and pressure wave device, combined device, or multi-mode intravascular treatment device). In the example shown in FIG. 1, the combined laser atherectomy and pressure wave device 102 includes a flexible elongate member 115 comprising a guidewire lumen 236, laser catheter 105, and sheath 103. In some aspects, the flexible elongate member is guided through the vasculature of the patient by a guidewire 118. In an example, the guidewire 118 is stationary within the patient's vasculature, and the flexible elongate member 115 is slid over the guidewire via the guidewire lumen until the distal end of the flexible elongate member has reached the stenosis within the vessel of interest.

The combined laser atherectomy and pressure wave device includes the features of both an intravascular laser atherectomy device and an intravascular pressure wave generating device or lithotripsy device, but with a sealed distal end such that no photoreactive liquid is introduced into the bloodstream of the patient. The composition and structure of the laser catheter 105 and sheath 103 may for example be similar to those described in U.S. Pat. Nos. 11,058,492 and 11,246,659, each of which is incorporated by reference as though fully set forth herein.

In the example shown in FIG. 1, the light source 140 is in optical communication with the laser catheter 105 by an optical communication line 145, such as an optical fiber or bundle of optical fibers. Similarly, the pump 150 is in fluid communication with the pressure wave fluid source 170, transparent fluid source 160, and reservoir 155, via fluid channels 180, and the pressure wave fluid source 170 and transparent fluid source 160 are in fluid communication with the sheath 103 via fluid conduits 180.

Depending on the implementation, the pressure wave fluid may be a liquid, a gas, or combinations thereof. Similarly, the transparent fluid may be a liquid or a gas, or combinations thereof. It is understood that the pressure wave fluid and the transparent fluid are different from one another.

In order to provide the benefits of laser atherectomy and pressure wave generation, without the need for precise repositioning of the laser catheter relative to the sheath and without introducing the pressure wave fluid or photoreactive fluid into the bloodstream of the patient, the combined laser atherectomy and pressure wave device 102 provides a laser catheter and sheath that are fixed or stationary relative to one another. Furthermore, the distal end of the combined laser atherectomy and pressure wave device 102 is sealed, such that the fluids used by the device (e.g., the pressure wave fluid and transparent fluid) remain inside the distal end of the device (although, depending on the implementation, they may exit at or near the proximal end of the flexible elongate member and be received by the reservoir 155).

It is understood that block diagrams are provided herein for exemplary purposes; a person of ordinary skill in the art will recognize myriad variations that nonetheless fall within the scope of the present disclosure. For example, block diagrams may show a particular arrangement of components, modules, services, steps, processes, or layers, resulting in a particular flow of data, light, fluids, etc. It is understood that some embodiments of the systems disclosed herein may include additional components, that some components shown may be absent from some aspects, and that the arrangement of components may be different than shown, resulting in different data flows while still performing the methods described herein.

Before continuing, it should be noted that the examples described above are provided for purposes of illustration and are not intended to be limiting. Other devices and/or device configurations may be utilized to carry out the operations described herein.

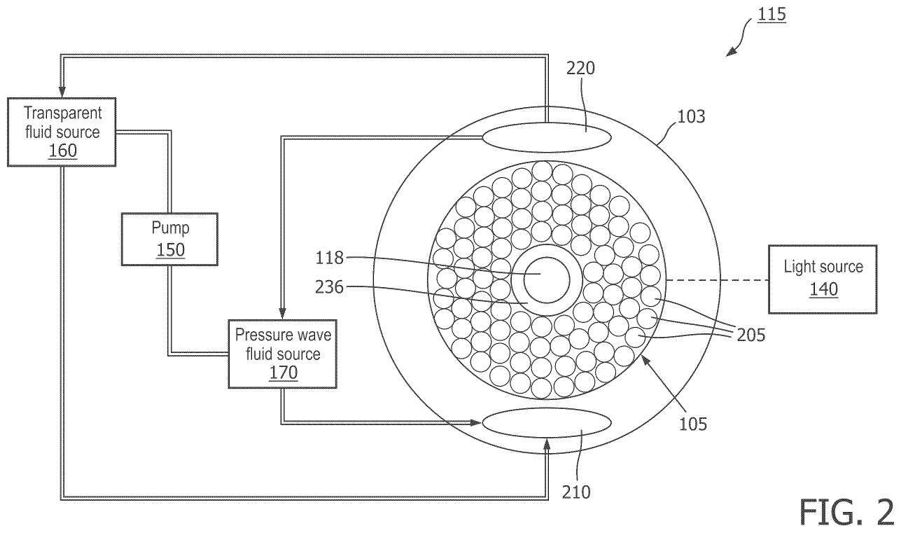

FIG. 2 is a diagrammatic, schematic, lateral cross-sectional view of at least a portion of an example combined laser atherectomy and pressure wave device 102, in accordance with at least one aspect of the present disclosure. Visible are the sheath 103, laser catheter 105, guidewire lumen 236, and guidewire 118, along with the light source 140, transparent fluid source 160, pump 150, and pressure wave fluid source 170.

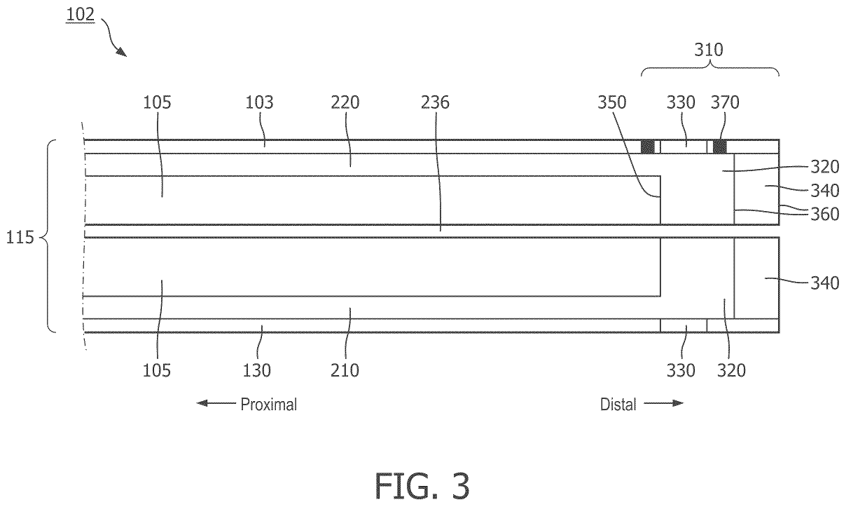

The laser catheter 105 includes one or multiple optical fibers 205. In some instances, the laser catheter can be referred to as optical fiber 105. The optical fiber and/or laser catheter can include an optical fiber core, optical fiber cladding, protective coating around the optical fiber(s), outer jacket around the optical fiber(s), etc. The laser catheter 105 and/or the optical fiber include the guidewire lumen 236 that receives a guidewire. The device 102 can move within the blood vessel along the guidewire. The laser catheter 105 is surrounded by the sheath 103, and may for example be physically coupled (e.g., adhered) to the sheath 103, or may be held in place by friction, etc. In the example shown in FIG. 3, the sheath 103 includes an inflow fluid channel 210 that allows fluid to enter a fluid chamber (e.g., a cavity between the proximal part of the laser catheter and the exit window lens system) as shown below in FIG. 3. During the laser atherectomy portion of a procedure, under the influence of a pump 150, this inflow fluid channel 210 may receive a transparent fluid (e.g., water, saline, air, or other gasses) from the transparent fluid source 160. The transparent fluid can then be expelled through an outflow fluid channel 220, such that it returns to the transparent fluid source 160 (or, alternatively, is received in an effluent reservoir as shown above). During the pressure wave generation portion of the procedure, the transparent fluid is displaced by a photoreactive pressure wave fluid (e.g., X-ray contrast fluid or other photoreactive fluid) from the pressure wave fluid source 170, which can be used to generate shockwaves as described below.

As a result, the system functions as an atherectomy device when water is in the cavity and as a shockwave device when contrast fluid is in the cavity. No mechanical movements of the laser catheter with respect to the sheath is required for this. To avoid blood entering the cavity containing the contrast for shockwave production is closed with an acoustic window for the shockwaves. In most aspects, the inflow fluid channel 210 is in fluid communication with the outflow fluid channel 220, but not with the guidewire lumen 236. It is understood that the size, shape, and positioning of the inflow fluid channel 210 and the outflow fluid channel 220 within the sheath 103 may be different than shown in FIG. 2.

FIG. 3 is a schematic, diagrammatic, longitudinal cross-sectional view of at least a portion of an example combined laser atherectomy and pressure wave device 102, in accordance with at least one aspect of the present disclosure. Visible is the flexible elongate member 115, including the sheath 103, laser catheter 105, guidewire lumen 236, fluid inflow channel 210, and fluid outflow channel 220.

A distal portion 310 of the sheath 103 includes a lens 340 (e.g., an exit lens, exit window, etc.) that allows light exiting the laser catheter 105 to leave the exit lens/window with sufficient fluence for laser atherectomy. Hence the distance between the laser catheter exit 350 and the exit lens/window 340 may typically be 5 mm or less. As a result, the light distribution at the exit lens/window 340 is only minorly diluted, such that it allows atherectomy functionality at the exit lens/window 340. The sheath 103 surrounding the laser catheter 105 includes a fluid inflow channel 210 that allows fluid to enter the cavity 320 (also referred to as a reaction chamber or fluid chamber) between the exit or distal end 350 of the laser catheter 105 and the exit lens/window. When this fluid chamber 320 is filled with a transparent fluid such as water or saline, the light is not absorbed and exits the exit lens/window 340 and can be used for the atherectomy function. The transparent fluid may exit the fluid chamber 320 through the outflow fluid channel 220. The transparent fluid may for example limit refraction mismatch at the interface of the laser catheter exit 350 with the fluid chamber 320, and of the fluid chamber 320 with the exit lens/window 340, in order to minimize unwanted reflection. In some aspects, the transparent fluid may also serve as a coolant, to prevent damage to the laser catheter 105, exit lens/window 340, sheath 103, or other components.

When the transparent fluid is replaced by an (opaque or translucent) photoreactive fluid (e.g., X-ray contrast liquid), activation of the laser catheter 105 can generate shockwaves (e.g., pressure waves of highly variable pressure) within the fluid chamber 320. The sheath 103 includes one or more acoustic windows 330, adjacent to the fluid chamber 320. These acoustic windows 330 may for example be made of an acoustically transparent material such as a polymer or hydrogel which has similar speed of sound to the fluid medium, which allows shockwaves to pass through, while preventing vapor bubbles, photoreactive liquid, or reaction products from exiting the flexible elongate member 115. As a result, the system functions as an atherectomy device when transparent liquid is in the fluid chamber and as a shockwave generation device when the photoreactive liquid is in the cavity. No mechanical movements of the laser catheter with respect to the sheath is required for this. To avoid blood entering the sheath 103, the distal portion 310 of the sheath 103 is sealed by the acoustic windows 330 and exit lens/window 340. That is, the structure of the sheath 103, the laser catheter 105, the acoustic window 330, and/or the lens 340 prevents that is inside the sheath 103 from exiting the sheath. In that regard, sheath 103, the laser catheter 105, and/or the acoustic window 330 can be a material (e.g., polymer) that is impermeable to fluid. In some instances, the lens 340 can be coupled to the sheath 103 at a distal end with an adhesive such that the adhesive and the lens 340 together provide the fluid seal at the distal end of the sheath. In some instances, the sheath 103 includes a fluid seal gasket (e.g., around the lens 340, immediately proximal to/distal to the lens 340), such that the fluid seal gasket and the lens 340 together provide the fluid seal at the distal end of the sheath. In some instances, the device 102 includes a sealed lens assembly (e.g., one or multiple lens, with fluid seal, such as gasket or adhesive, with coupling between one or multiple lens to the sheath 103, such as adhesive).

The exit lens system 340 can be a flat exit window or may include one or more curved surfaces that have optical power. The lens surfaces preferably have an anti-reflex coating 360 to minimize light loss and to reduce damage to the laser catheter 105 and other components due to the reflection of the lens surfaces. In some embodiments, the exit lens/window 340 may be made of a polymer material. However, polymer materials may be opaque to UV light and/or easily damaged by high-intensity laser light, whereas UV light or high-intensity laser light may be favorable for triggering reactions in a photoreactive liquid. Therefore, in some aspects, the exit lens/window 340 is made of a transparent ceramic material such as glass or fused silica.

Thus, the fluid is completely sealed within volume inside sheath 103 (inflow channel 210 and outflow channel 220 and fluid chamber 320). No fluid enters the ambient environment (e.g., the blood vessel, the blood inside the blood vessel). This is advantageous because some patients can have adverse reactions to fluids like radiopaque contrast. The presence of the guidewire lumen 236 does not adversely impact the fluid seal. The fluid chambers 210, 220, 320 can be positioned around the guidewire lumen 236 (e.g., volume(s) of the sheath 103 and/or laser catheter 105 that are separate from the guidewire lumen 236). In some aspects, the sheath 103 and laser catheter 105 are physically or mechanically coupled (e.g., components of the sheath are stationary relative to components of the laser catheter and vice versa). In some aspects, no translational or rotational movement of laser catheter 105 relative to sheath 103 (or vice versa) occurs. In those aspects, no such movement required in order to operate the device in either atherectomy mode or pressure wave generation mode.

In some aspects, radiopaque markers 370 may be used to mark the locations of the acoustic windows such that their locations can be seen on X-ray fluoroscopy.

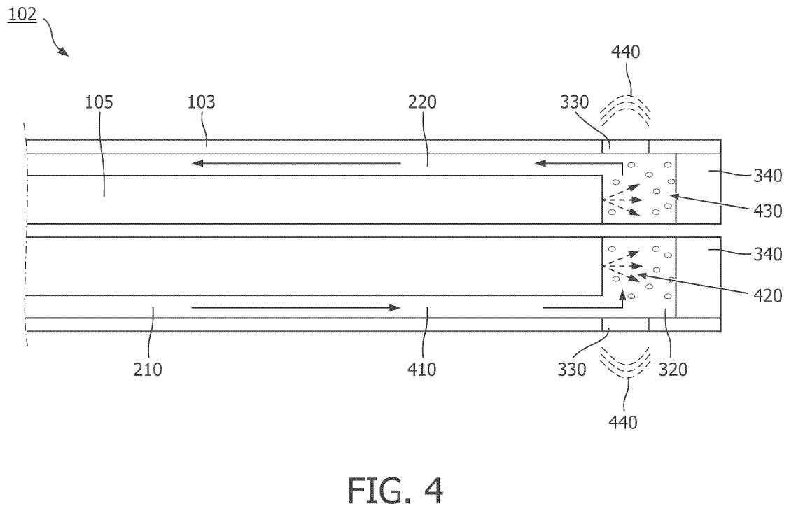

FIG. 4 is a schematic, diagrammatic, longitudinal cross-sectional view of at least a portion of an example combined laser atherectomy and pressure wave device 102, in accordance with at least one aspect of the present disclosure. Visible are the sheath 103, laser catheter 105, exit lens/window 340, fluid inflow channel 210, fluid chamber 320, acoustic windows 330, and fluid outflow channel 220.

In the example shown in FIG. 4, the fluid inflow channel 210, fluid outflow channel 220, and fluid chamber 320 are filled with a photoreactive liquid or pressure wave fluid 410. In a non-limiting example, some X-ray contrast fluid produces an exothermic reaction when struck by ultraviolet light (e.g., light with a wavelength of between 100 nm and 400 nm, and preferably between 300 nm and 400 nm), and can thus serve as a photoreactive liquid 410. Thus, when the laser catheter 105 emits laser light 420 into the photoreactive liquid 410, the laser light produces chemical or physical reactions 430 that can generate lithotripsy shockwaves or pressure waves 440 by, for example, producing rapidly expanding gas bubbles. The shockwaves or pressure waves 440 can then pass from the fluid chamber 320 into the exterior environment (e.g., the blood moving within a blood vessel) via the acoustically transparent windows 330. It is noted that in the example shown in FIG. 4, shockwaves or pressure waves 440 are being emitted laterally, in a radial direction relative to the sheath 103. Depending on the configuration of the acoustic windows 330, the shockwaves or pressure waves may be emitted on one side only, on one side and another side (whether opposite or otherwise), through an annular ring fully encircling the sheath 103, or combinations thereof. The laser light 420 can be one pulse or beam (e.g., a continuous pulse/beam) or a series of pulses or beams (e.g., multiple separate pulses/beams). In some instances the pressure waves 440 are generated by a portion of the light emitted by the optical fiber 105.

Examples of contrast fluids can be found in U.S. Pat. Nos. 11,058,492 and 11,246,659.

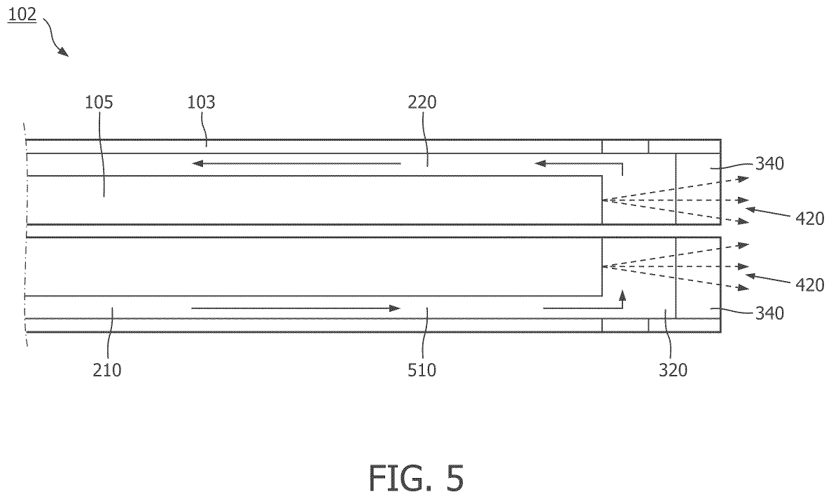

FIG. 5 is a schematic, diagrammatic, longitudinal cross-sectional view of at least a portion of an example combined laser atherectomy and pressure wave device 102, in accordance with at least one aspect of the present disclosure. Visible are the sheath 103, laser catheter 105, exit lens/window 340, fluid inflow channel 210, fluid chamber 320, acoustic windows 330, and fluid outflow channel 220. In the example shown in FIG. 5, the fluid inflow channel 210, fluid outflow channel 220, and fluid chamber 320 are filled with a transparent fluid 510 such as water, saline, or air, such that laser light 420 exiting the laser catheter 105 can pass unimpeded to the exit window 340. It is noted that the laser light 420 diverges after exiting from the laser catheter, and due to the index of refraction of the exit window 340, the laser light 420 diverges still further during and after its passage through the exit window 340. This divergence decreases the power per unit area of the laser light 420, such that it may only be effective at ablating vascular plaques or other invasive materials within a limited range. In an example, this range may be on the scale of microns to millimeters from the tip of the device. Thus, the exit window 340 must be placed relatively close to the plaque material for the atherectomy procedure to be successful. The laser light 420 can be one pulse or beam (e.g., a continuous pulse/beam) or a series of pulses or beams (e.g., multiple separate pulses/beams). In some instances, a portion of the light emitted by the optical fiber 105 is transmitted to outside of the device 102.

Thus, it can be seen that the combined laser atherectomy and pressure wave device 102 includes an outer sheath, a laser catheter fixedly positioned within the sheath, a sealed lens (e.g., a sealed exit lens) at the distal end of the outer sheath, and chamber that is distal of the laser catheter and proximal of the exit lens. The chamber can serve as both a fluid chamber and a transmission chamber, which is in fluid communication with the sealed acoustic windows. The sealed acoustic window and sealed lens (e.g., the sealed exit lens) prevent the photoreactive fluid from entering the blood vessel or other body lumen. The guidewire lumen, while open at both ends, is sealed against the exit lens such that it does not affect the sealing of the fluid chamber or exit lens. In some instances, filling the chamber with the photoreactive fluid includes removing the transparent fluid from the chamber via the outflow channel. In some instances, filling the chamber with the transparent fluid includes removing the photoreactive fluid from the chamber via the outflow channel. In some aspects, one fluid may serve both functions. In such cases, the fluid may be optically transmissive (e.g., translucent, forward-scattering, etc.) without being transparent per se. In such an arrangement, when the chamber is filled with the single fluid, a single beam of laser light exiting the laser catheter causes a portion of the beam of laser light to be transmitted through the lens (e.g., in a forward or distal direction, and also causes the photoreactive fluid to generate a shockwave or pressure wave that is transmitted radially outward from the sheath via the acoustic window). In some instances, a single fluid may be photoreactive to one wavelength/frequency of laser light and transparent to a different wavelength/frequency of laser light, such that a single fluid may serve as both the photoreactive fluid and the transparent fluid. In that regard, the processor circuit 106 and/or the user can control the light source 140 to provide one wavelength/frequency of light to the optical fiber 105 (with the single fluid inside the sheath 103) for laser atherectomy (this wavelength/frequency of light is transmitted through the fluid to outside of the device 102) and a different wavelength/frequency of light to the optical fiber 105 (with the single fluid inside the sheath) to generate pressure waves (this wavelength/frequency of light interacts with the fluid to generate pressure waves that propagate outside of the device 102). The different wavelengths/frequencies of light can be selectively provided to the optical fiber 105 at different times.

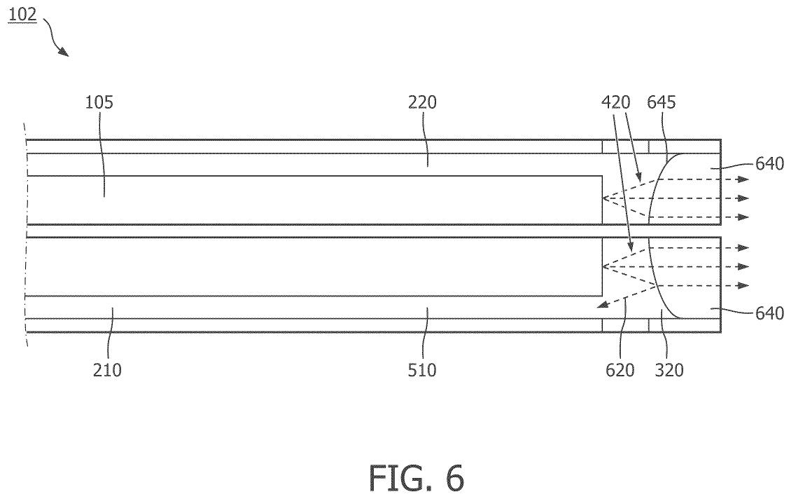

FIG. 6 is a schematic, diagrammatic, longitudinal cross-sectional view of at least a portion of an example combined laser atherectomy and pressure wave device 102, in accordance with at least one aspect of the present disclosure. Visible are the sheath 103, laser catheter 105, fluid inflow channel 210, fluid chamber 320, acoustic windows 330, fluid outflow channel 220, and transparent fluid 510.

In the example shown in FIG. 6, the flat exit window of FIGS. 3-5 has been replaced with an exit lens 640 that includes a convex inner surface. When laser light 420 strikes the exit lens 640, the convex inner surface 645 causes reflected laser light 620 to diverge, thus reducing the chance of its damaging the laser catheter 105 or other components. The convex inner surface 645 also causes the exit lens 640 to at least partially undo the divergence of the laser light 420 that passes through the exit lens 640, such that the exiting laser light 420 is parallel, or more parallel than in the example shown in FIG. 5. This may improve the effective range of the laser catheter 105 for the laser atherectomy portion of a procedure, such that precise positioning of the combined laser atherectomy and pressure wave device 102, relative to the stenosis, is less important.

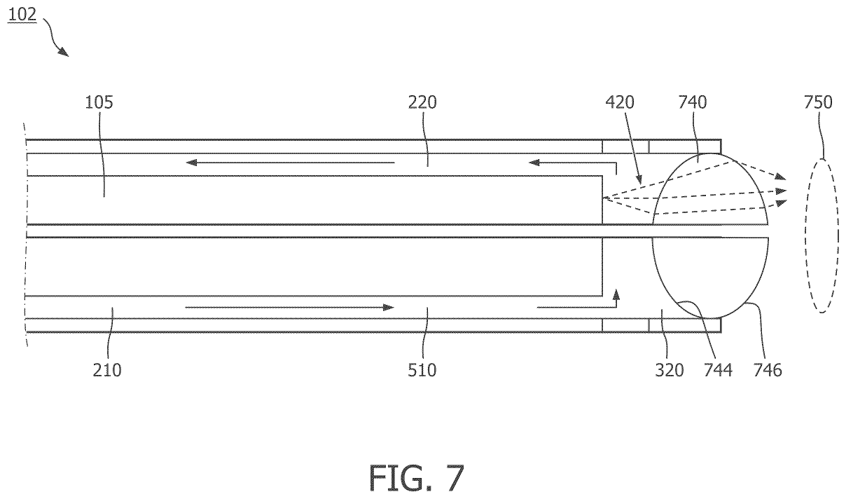

FIG. 7 is a schematic, diagrammatic, longitudinal cross-sectional view of at least a portion of an example combined laser atherectomy and pressure wave device 102, in accordance with at least one aspect of the present disclosure. Visible are the sheath 103, laser catheter 105, fluid inflow channel 210, fluid chamber 320, acoustic windows 330, fluid outflow channel 220, and transparent fluid 510.

In the example shown in FIG. 7, the flat exit window of FIGS. 3-5 has been replaced with an exit lens 740 that includes a convex inner surface 744 and a convex outer surface 746. When laser light 420 strikes the exit lens 740, the convex inner surface 744 causes reflected laser light to diverge, as described above. Furthermore, the convex inner surface 744 and convex outer surface 746 also cause the exit lens 740 to undo the divergence of the laser light 420 that passes through the exit lens 740, such that the exiting laser light 420 can be reconverged toward a focal point or focal ring 750 given large enough optical power of the focal lens 740. With proper placement relative to the stenosis, this arrangement may improve the power per unit area of the laser catheter 105 for the laser atherectomy portion of a procedure, such that better or faster ablation is possible.

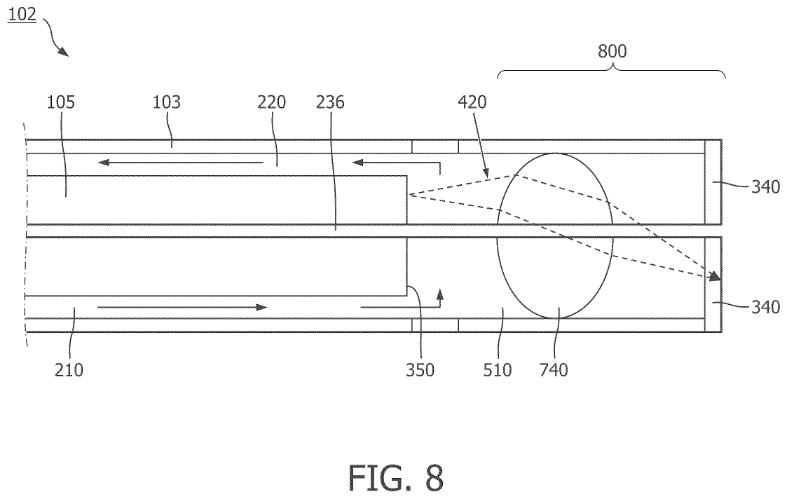

FIG. 8 is a schematic, diagrammatic, longitudinal cross-sectional view of at least a portion of an example combined laser atherectomy and pressure wave device 102, in accordance with at least one aspect of the present disclosure. Visible are the sheath 103, laser catheter 105, fluid inflow channel 210, fluid chamber 320, acoustic windows 330, fluid outflow channel 220, and transparent fluid 510.

In the example shown in FIG. 8, the lens system is a relay lens system 800 that includes both a convex-convex lens 740 and a flat exit lens/window 340, such that lens 740 images the exit 350 the laser catheter 105 just in front of the exit window. It is noted that this arrangement may be similar to the operation of an endoscope, in which the object in front of the endoscope is relayed to the eyepiece by a relay lens system. Examples can be found in U.S. Pat. No. 5,097,359, which is incorporated by reference as though fully set forth herein. The lens materials are chosen such that they are transparent for the laser light produced by the console and anti-reflex coatings are applied to reduces the reflections below the limit where they can harm the laser catheter. In particular, the system is suitable for the 308 nm wavelength produced for example by the CVX-300: 308 nm ultraviolet laser. An excimer laser or other type of laser may be used instead or in addition. One, the other, or both of the lens 740, 340 can provide fluid seal (e.g., adhesive, gasket, etc.) to prevent fluid that is inside the sheath 103 from leaving the device 103 (e.g., into the blood vessel).

One advantage of the arrangement shown in FIG. 8 is that placing the exit window 340 against plaque material or other portions of the stenosis is functionally equivalent to placing the exit or distal end 350 of the laser catheter 105 against the same tissue, and thus the operation of the combined laser atherectomy and pressure wave device 102 in atherectomy mode may be much the same for the clinician as operating a naked laser catheter 105 without a sheath 103, or extended beyond the end of the sheath 103.

It is noted that the refractive index of the pressure wave fluid, photoreactive fluid, or contrast liquid can be used alter the functionality of the relay lens system 800. It is noted that if there is a significant difference in refractive index between the two fluids, then when the refractive index of the pressure wave fluid is lower than that of the transparent fluid, then the relay lens image is formed before the exit window. Hence, before the light reaches the exit window it is substantially diluted and no longer suitable for atherectomy functionality. However, if the contrast liquid has the same refractive index as the transparent fluid (e.g., water or saline) then the light that is not absorbed by the contrast liquid will focus at the exit window so that both functionalities i.e., atherectomy and shockwave production, can take place at the same time.

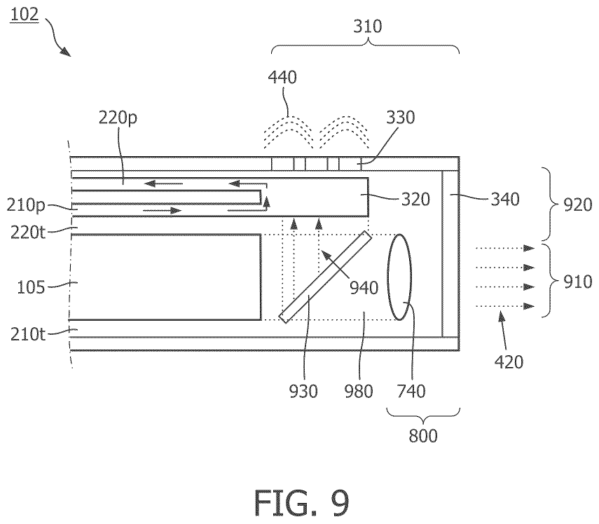

FIG. 9 is a schematic, diagrammatic, longitudinal cross-sectional view of at least a portion of an example combined laser atherectomy and pressure wave device 102, in accordance with at least one aspect of the present disclosure. Visible are the sheath 103, laser catheter 105, fluid inflow channel 210, fluid chamber 320, acoustic windows 330, fluid outflow channel 220, and transparent fluid 510. However, in the example shown in FIG. 9, the combined laser atherectomy and pressure wave device 102 employing a lens system 800 (including the convex-convex lens 740 and flat exit window 340) and a beam splitter 920. The lens system 800 may be or include a relay lens.

When using a partial beam splitter 930 angled at (for example) 45 degrees in front of the distal end of the laser, a first portion 420 of the laser light produced by the laser catheter is directed through the transmission chamber 980 and the sealed lens 800, and can thus be used for the atherectomy, while a second portion 940 of the laser light can be used for the shockwave generation. When the photoreactive fluid inlet channel 210p, fluid chamber 320, and photoreactive fluid outlet channel 220p are placed outside the beam 420 for the atherectomy, the absorption by the photoreactive liquid will have no further influence on the atherectomy function. The transparent fluid can either be present all the time or can then be delivered through a separate inlet channel 210t and outlet channel 220t.

This arrangement creates a shockwave or pressure wave generation portion 920 of the distal portion 310 of the sheath, and a laser atherectomy portion 910 of the distal portion 310 of the sheath. The shockwave or pressure wave generation portion 920 includes not only the fluid chamber 320 but also one or more acoustically transparent windows 330 to allow passage of shockwaves or pressure waves 440. In this arrangement, shockwaves or pressure waves are emitted from only a portion of the circumference of the combined laser atherectomy and pressure wave device 102.

In some aspects, the beam splitter is a polarizing beam splitter such that, depending on the polarization of the laser light, either the atherectomy functionality or the shockwave functionality is enabled. For instance, this switching can be achieved by controlling the polarization of the light entering the laser catheter 105. In such aspects, it may be desirable for the fibers inside the laser catheter to be polarization-maintaining fibers. In other aspects, a non-polarizing beam splitter may enable the atherectomy and shockwave generation functions to happen simultaneously. It is noted that the beam splitter 930 and lens arrangement 800 are coupled to the sheath 103.

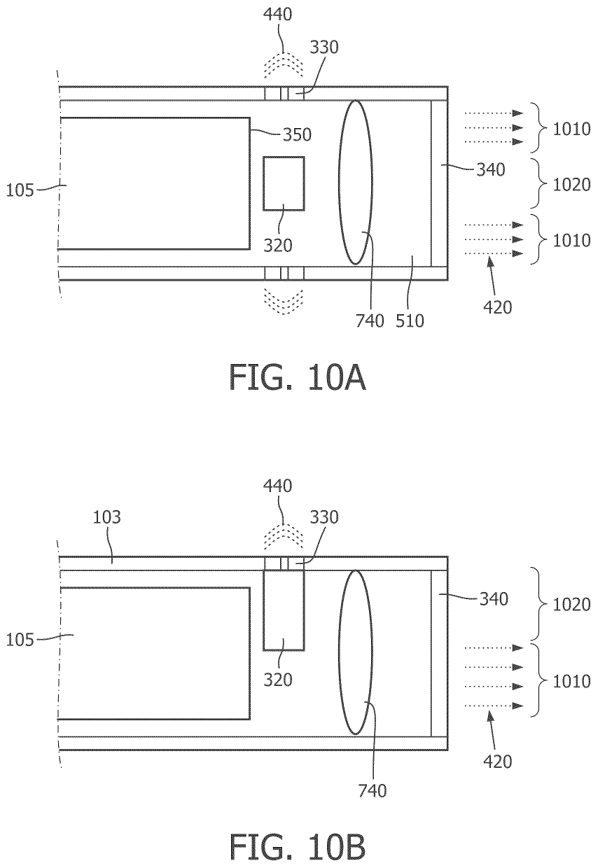

FIG. 10A is a schematic, diagrammatic, longitudinal cross-sectional view of at least a portion of an example combined laser atherectomy and pressure wave device 102, in accordance with at least one aspect of the present disclosure. In the example shown in FIG. 10A, the fluid chamber 320 covers only part of the laser catheter exit surface 350, and functions as a diaphragm. Transparent fluid 510 surrounding the fluid chamber 320 can transmit pressure waves or shockwaves 440 to the acoustic windows 330 for transmission to the exterior of the sheath. Photoreactive fluid inlet and outlet channels may carry photoreactive fluid into and out of the fluid chamber as shown for example in FIGS. 4 and 9. In this aspect, transmission portions 1010 of the transmission window 340 can be used for atherectomy (for instance an annulus shape), whereas an occluded portion 1020 (e.g., a central portion) is blocked by the fluid chamber 320.

FIG. 10B is a schematic, diagrammatic, longitudinal cross-sectional view of at least a portion of an example combined laser atherectomy and pressure wave device 102, in accordance with at least one aspect of the present disclosure. The configuration shown in FIG. 10B is similar to that shown in FIG. 10A, except that the fluid chamber 320 is located off-center and is in direct contact with the sheath 103 and the acoustic windows 330. In this case, the transmission portions 1010 and occluded portions 1020 of the exit window 340 are shifted accordingly. It is understood that the fluid chamber 320 can be positioned at any suitable location within the combined laser atherectomy and pressure wave device 102.

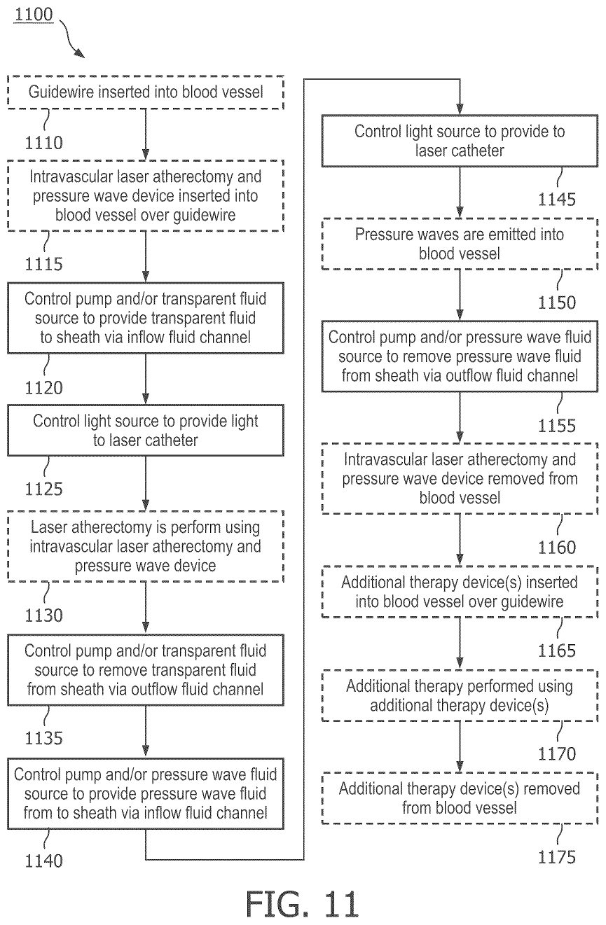

FIG. 11 shows a flow diagram of an example laser atherectomy and calcified stenosis fracturing method 1100, in accordance with at least one aspect of the present disclosure. It is understood that the steps of method 1100 may be performed in a different order than shown in FIG. 11, additional steps can be provided before, during, and after the steps, and/or some of the steps described can be replaced or eliminated in other aspects. One or more of steps of the method 1100 can be carried out at least in part by one or more devices and/or systems described herein, such as components of the intravascular treatment system 100, processor circuit 106, and/or processor circuit 2250.

In step 1110, the method 1100 includes inserting the guidewire into the target blood vessel and advancing it to, or through, the target stenosis.

In step 1115, the method 1100 includes inserting the intravascular combined laser atherectomy and pressure wave device into the target blood vessel over the guidewire, and advancing it until it is in proximity to the stenosis.

In step 1120, the method 1100 includes controlling the pump and/or transparent fluid source to provide transparent fluid into to distal portion of the sheath via the inflow fluid channel.

In step 1125, the method 1100 includes controlling the light source to provide light (e.g., ultraviolet laser light) to the laser catheter.

In step 1130, the method 1100 includes performing laser atherectomy on the blood vessel by using laser light, emitted by the intravascular combined laser atherectomy and pressure wave device, to ablate plaque or other sources of blockage.

In step 1135, the method 1100 includes controlling the pump and/or transparent fluid source to remove the transparent fluid from the distal portion of the sheath via the outflow fluid channel.

In step 1140, the method 1100 includes controlling the pump and/or pressure wave fluid source to provide pressure wave fluid (e.g., photoreactive liquid or X-ray contrast fluid) to the distal portion of the sheath via the inflow fluid channel.

In step 1145, the method 1100 includes controlling the light source to provide light (e.g., UV laser light) to the laser catheter.

In step 1150, the method 1100 includes emitting shockwaves or pressure waves into the walls of the blood vessel. These shockwaves are a consequence of the laser light interacting with the pressure wave fluid or photoreactive fluid, and are transmitted laterally/radially from the sheath via the acoustically transparent windows. The shockwaves or pressure waves can be used to fracture calcifications embedded in the vessel wall.

In step 1155, the method 1100 includes controlling the pump and/or pressure wave fluid source to remove the pressure wave fluid or photoreactive fluid from the distal portion of the sheath via the outflow fluid channel.

In step 1160, the method 1100 includes removing the intravascular combined laser atherectomy and pressure wave device from the blood vessel by reversing it over the guidewire.

In step 1165, the method 1100 includes inserting additional therapy device(s) inserted into the blood vessel by guiding them over the guidewire. Such additional therapy devices may for example include balloon catheters or stents.

In step 1170, the method 1100 includes performing additional therapy using the additional therapy device(s). Such additional therapy may for example include balloon angioplasty and/or stent placement to dilate the vessel at the location of the fractured calcifications.

In step 1175, the method 1100 includes removing the additional therapy device(s) from the blood vessel, along with the guidewire. The method is now complete.

It is noted that steps 1120, 1125, 1135, 1140, and 1155 are, or can be, performed automatically by the console 130 (see FIG. 1), whereas steps 1110, 1115, 1130, 1145, 1150, 1160, 1165, 1170, and 1175 are consequences of the automatic steps or are at least partially manual steps that are performed by a clinician operating the intravascular treatment system 100 and combined laser atherectomy and pressure wave device 102.

It is noted that flow diagrams are provided herein for exemplary purposes; a person of ordinary skill in the art will recognize myriad variations that nonetheless fall within the scope of the present disclosure. For example, the logic of flow diagrams may be shown as sequential. However, similar logic could be parallel, massively parallel, object oriented, real-time, event-driven, cellular automaton, or otherwise, while accomplishing the same or similar functions. In order to perform the methods described herein, a processor may divide one or more of the steps described herein into a plurality of machine instructions and may execute these instructions at the rate of several hundred, several thousand, several million, or several billion per second, in a single processor or across a plurality of processors.

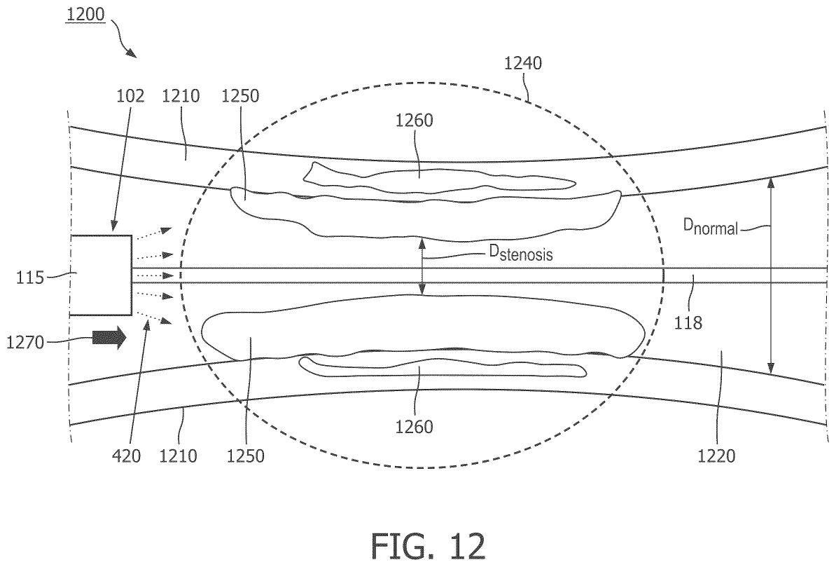

FIG. 12 is a schematic, diagrammatic, longitudinal cross-sectional view of at least a portion of blood vessel 1200 undergoing treatment by an example combined laser atherectomy and pressure wave device 102, in accordance with at least one aspect of the present disclosure. Visible are the flexible elongate member 115, guidewire 118, and laser light 420.

The blood vessel 1200 includes a vessel wall 1210 and a lumen 1220 with a normal inner diameter Dₙₒᵣₘₐₗ. The blood vessel also includes a stenosis 1240, which includes a plaque or other obstruction 1250 and a vessel wall calcification 1260. As a result of these features, the diameter of the lumen 1220 in the stenosis 1240 is Dₛₜₑₙₒₛᵢₛ, which is smaller than Dₙₒᵣₘₐₗ, and may in some cases be zero (e.g., in the case of a chronic total occlusion or CTO).

In order to treat the stenosis, the flexible elongate member 115 of the combined laser atherectomy and pressure wave device 102 is advanced along the guidewire 118 in a forward longitudinal direction 1270 along a longitudinal axis (e.g., along the length of the guidewire) while emitting laser light 420 in the forward longitudinal direction 1270, in order to perform the laser atherectomy portion of the procedure.

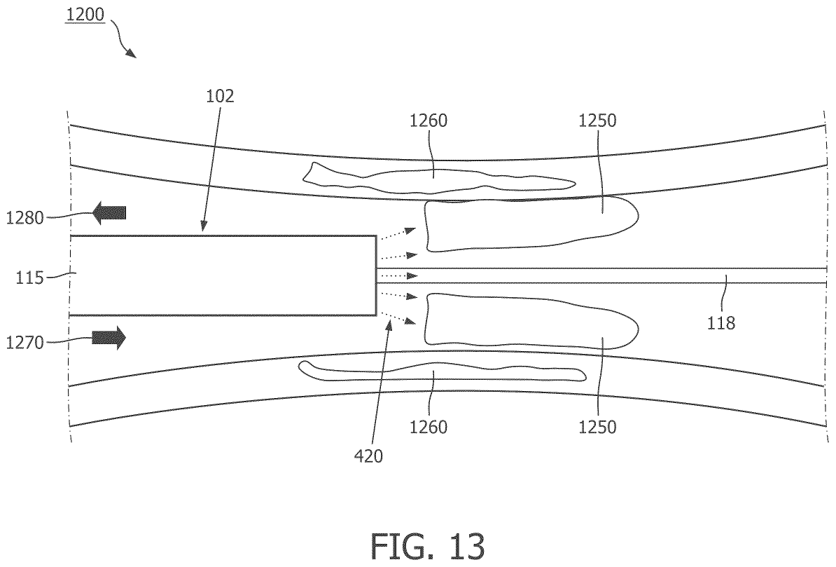

FIG. 13 is a schematic, diagrammatic, longitudinal cross-sectional view of at least a portion of blood vessel 1200 undergoing treatment by an example combined laser atherectomy and pressure wave device 102, in accordance with at least one aspect of the present disclosure. Visible are the flexible elongate member 115, guidewire 118, laser light 420, plaque 1250, and calcifications 1260. In the example shown in FIG. 13, the plaque 1250 has been partially ablated by the laser light 420 as the flexible elongate member 115 is advanced in the forward longitudinal direction. Once the plaque 1250 has been completely ablated and the flexible elongate member 115 has been advanced past the position of the plaque 1250, the flexible elongate member 115 is then pulled back in a reverse longitudinal direction 1280.

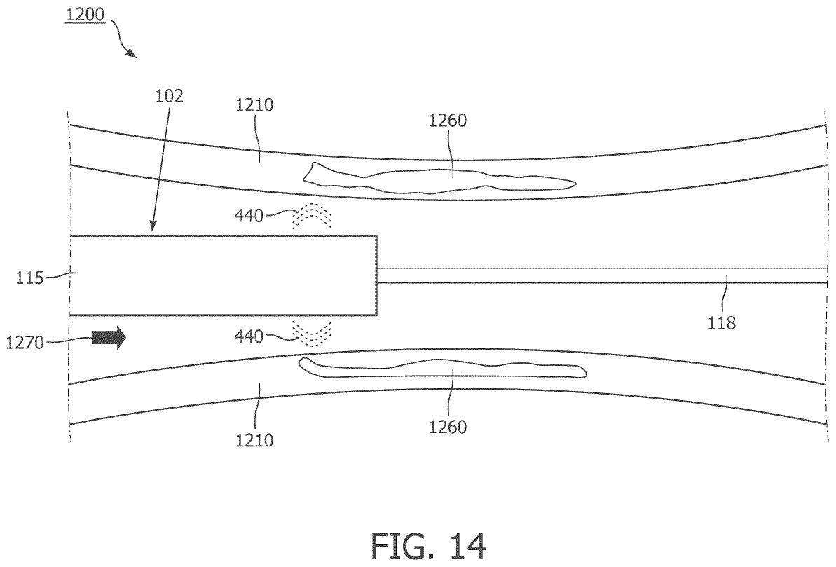

FIG. 14 is a schematic, diagrammatic, longitudinal cross-sectional view of at least a portion of blood vessel 1200 undergoing treatment by an example combined laser atherectomy and pressure wave device 102, in accordance with at least one aspect of the present disclosure. Visible are the flexible elongate member 115, guidewire 118, and calcifications 1260. In the example shown in FIG. 14, the plaque has been completely ablated by the laser light, and the flexible elongate member 115 has been pulled back to the most proximal portion of the stenosis. Now, in pressure wave or shockwave generation mode, the combined laser atherectomy and pressure wave device 102 is advanced through the stenosis again, while emitting shockwaves or pressure waves 440 in a radial direction, toward the vessel wall 1210. These shockwaves or pressure waves 440 can fracture the calcifications 1260 that are embedded in the soft tissue of the vessel wall 1210, without damaging the soft tissue of the vessel wall 1210. It is noted that the actions shown in FIG. 14 can also be performed during the pullback step.

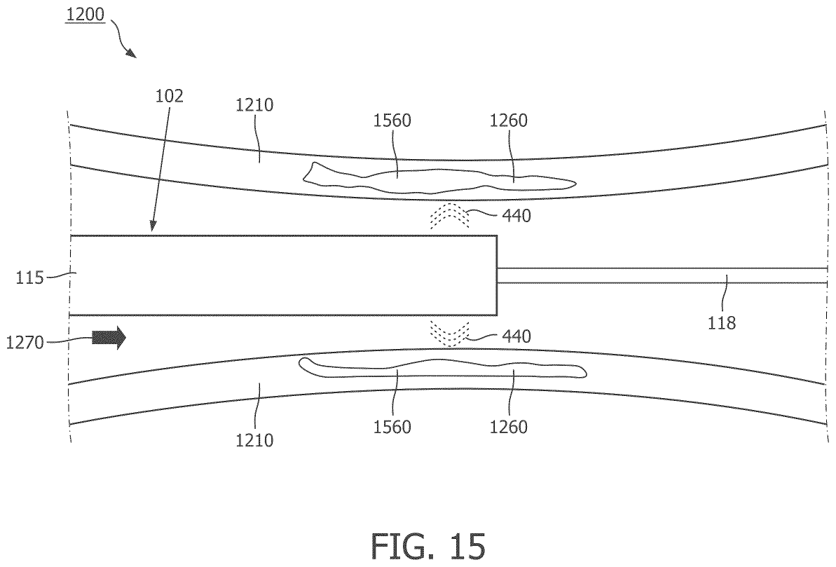

FIG. 15 is a schematic, diagrammatic, longitudinal cross-sectional view of at least a portion of blood vessel 1200 undergoing treatment by an example combined laser atherectomy and pressure wave device 102, in accordance with at least one aspect of the present disclosure. Visible are the flexible elongate member 115, guidewire 118, and calcifications 1260. In the example shown in FIG. 15, the solid calcification 1260 has been partially converted to fractured calcification 1560 by the shockwaves or pressure waves 440 (e.g., via lithotripsy) as the flexible elongate member 115 is advanced through the stenosis. Solid calcifications 1260 are generally resistant to dilation by balloon catheters or stents. However, fractured calcifications 1560 tend to be well retained by the soft tissue of the vessel wall, and yet capable of dilation.

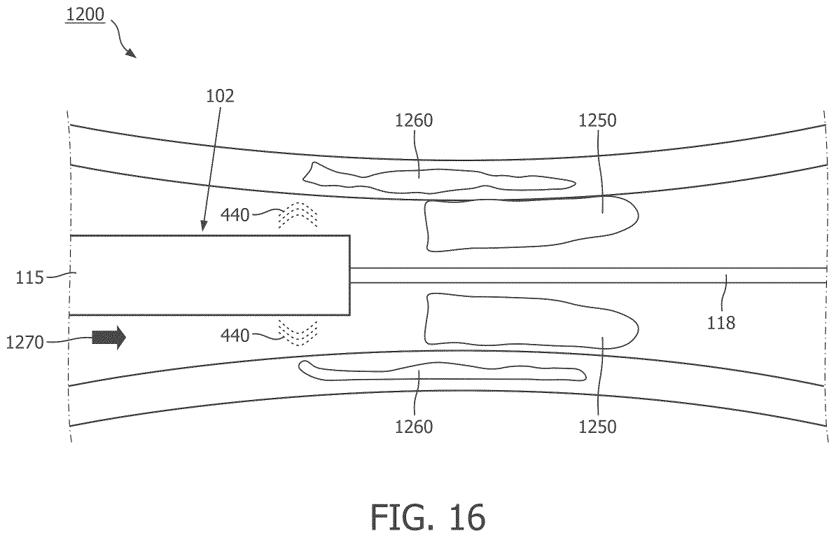

FIG. 16 is a schematic, diagrammatic, longitudinal cross-sectional view of at least a portion of blood vessel 1200 undergoing treatment by an example combined laser atherectomy and pressure wave device 102, in accordance with at least one aspect of the present disclosure. Visible are the flexible elongate member 115, guidewire 118, shockwaves or pressure waves 440, plaque 1250, and calcifications 1260. In the example shown in FIG. 16, the plaque 1250 has been partially ablated by the laser light as the flexible elongate member 115 is advanced along the guidewire 118 in the forward longitudinal direction 1270. However, in this instance, the combined laser atherectomy and pressure wave device 102 is switched to shockwave or pressure wave generation mode before the plaque 1250 has been completely ablated.

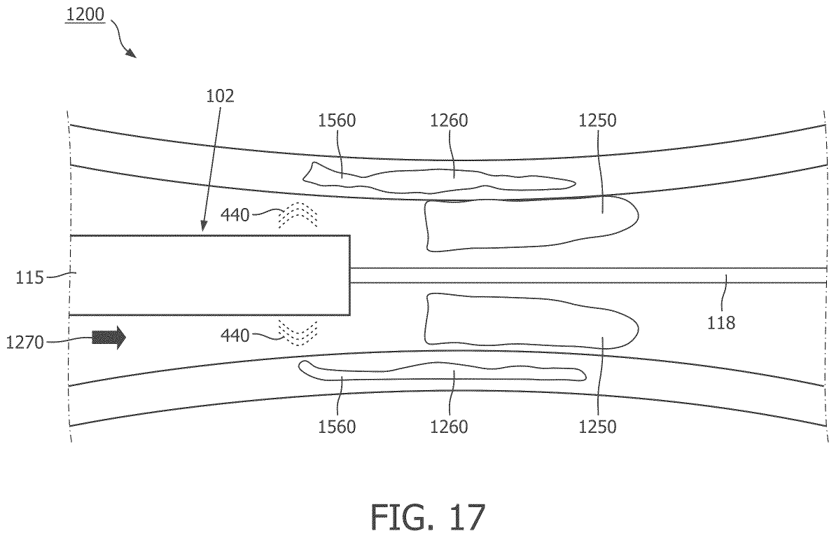

FIG. 17 is a schematic, diagrammatic, longitudinal cross-sectional view of at least a portion of blood vessel 1200 undergoing treatment by an example combined laser atherectomy and pressure wave device 102, in accordance with at least one aspect of the present disclosure. Visible are the flexible elongate member 115, guidewire 118, plaque 1250, and calcifications 1260. In the example shown in FIG. 15, the solid calcification 1260 has been partially converted to fractured calcification 1560 by the shockwaves or pressure waves 440 as the flexible elongate member 115 is advanced through the stenosis.

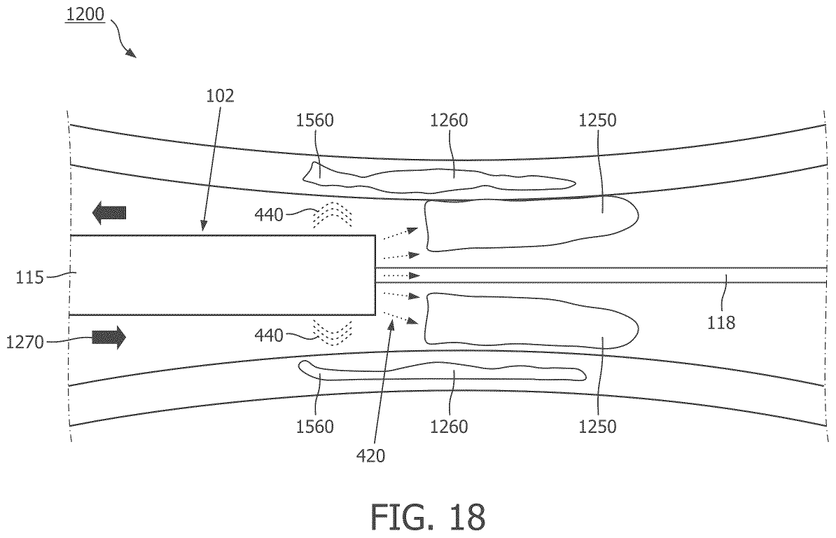

FIG. 18 is a schematic, diagrammatic, longitudinal cross-sectional view of at least a portion of blood vessel 1200 undergoing treatment by an example combined laser atherectomy and pressure wave device 102, in accordance with at least one aspect of the present disclosure. Visible are the flexible elongate member 115, guidewire 118, plaque 1250, calcifications 1260, laser light 420, and shockwaves or pressure waves 440. In the example shown in FIG. 18, the combined laser atherectomy and pressure wave device 102 is operating in simultaneous laser atherectomy and shockwave or pressure wave generation mode. This permits the plaque 1250 to be ablated at the same time the solid calcification 1260 is converted to fractured calcification 1560, while the flexible elongate member 115 is advanced along the guidewire 118 in the forward longitudinal direction.

Once the plaque 1250 has been entirely ablated and the calcification 1260 has been entirely converted to fractured calcification 1560, the flexible elongate member 115 can be pulled backward along the guidewire 118 in the reverse longitudinal direction and removed from the blood vessel 1200.

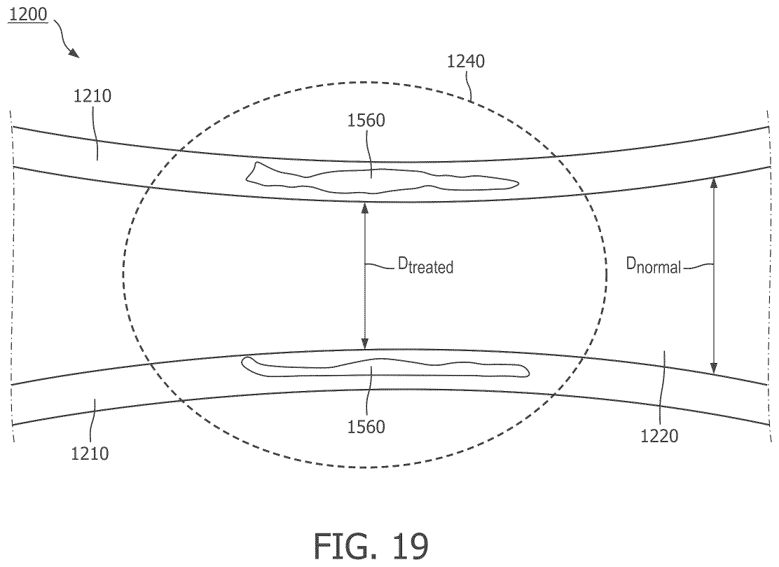

FIG. 19 is a schematic, diagrammatic, longitudinal cross-sectional view of at least a portion of blood vessel 1200 after treatment by a combined laser atherectomy and pressure wave device, in accordance with at least one aspect of the present disclosure. Visible are the vessel walls 1210, lumen 1220, and normal diameter Dₙₒᵣₘₐₗ. Following treatment by the combined laser atherectomy and pressure wave device, the stenosis now has a diameter D_{treated}, which is greater than Dₛₜₑₙₒₛᵢₛ but still less than Dₙₒᵣₘₐₗ.

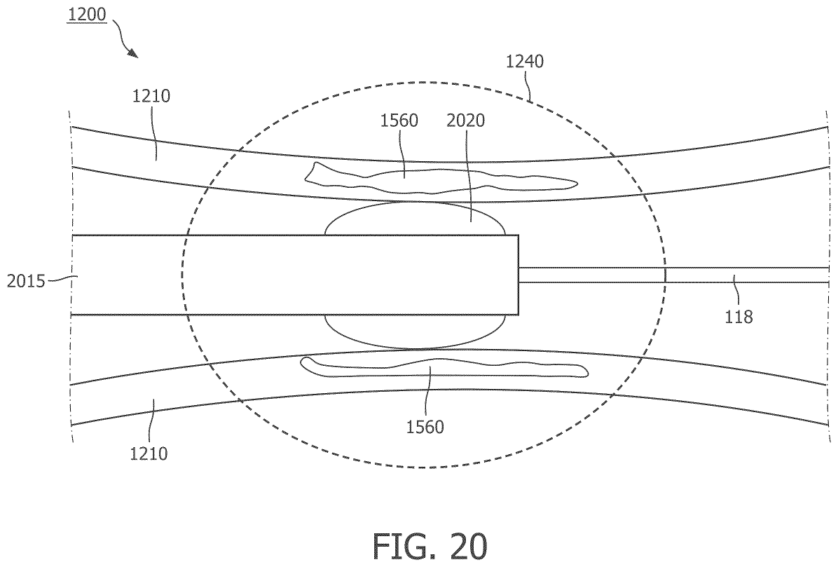

FIG. 20 is a schematic, diagrammatic, longitudinal cross-sectional view of at least a portion of blood vessel 1200 after treatment by a combined laser atherectomy and pressure wave device, in accordance with at least one aspect of the present disclosure. In the example shown in FIG. 20, a balloon catheter 2020 has been advanced along the guidewire and then inserted into the stenosis 1240 in order to dilate the blood vessel 1200 at the location of the stenosis 1240. Since the plaque has been removed and the solid calcification has been converted into fractured calcification 1560, the stenosis is now capable of being dilated/expanded by treatment devices such as the balloon catheter 2020.

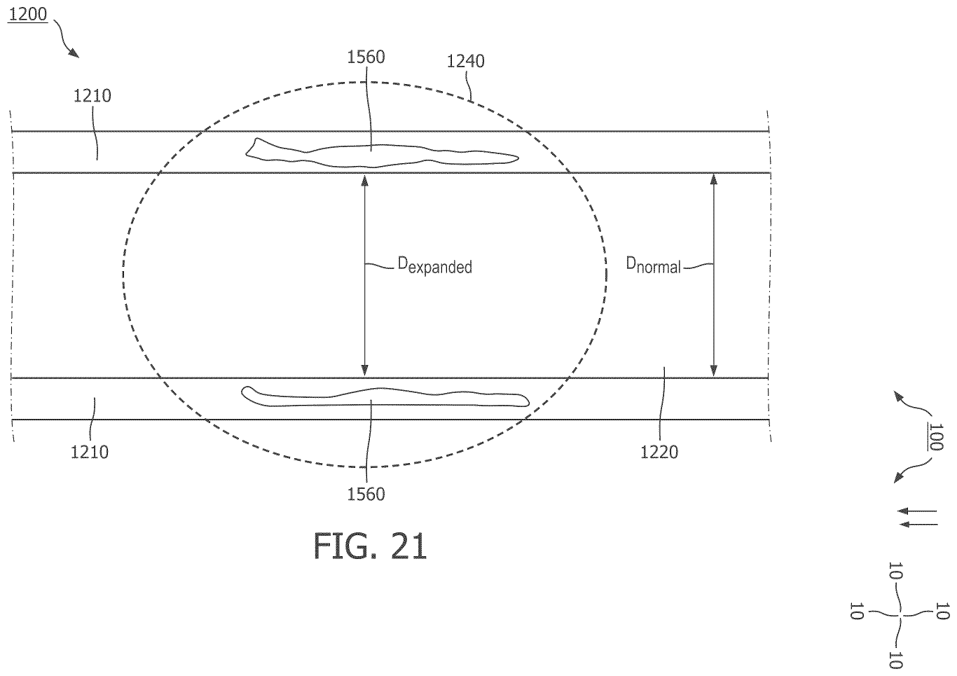

FIG. 21 is a schematic, diagrammatic, longitudinal cross-sectional view of at least a portion of blood vessel 1200 after treatment by a combined laser atherectomy and pressure wave device and a balloon catheter, in accordance with at least one aspect of the present disclosure. Following treatment with the balloon catheter, the stenosis 1240 has been expanded to a diameter D_{expanded}, which is greater than the diameters Dₛₜₑₙₒₛᵢₛ (see FIG. 12) or D_{treated} (see FIG. 19), and may be equal or comparable to Dₙₒᵣₘₐₗ. In some cases, treatment of the stenosis may now be complete. In other cases, a stent may be placed, or other interventions may be taken, to ensure the blood vessel 1200 maintains a healthy diameter.

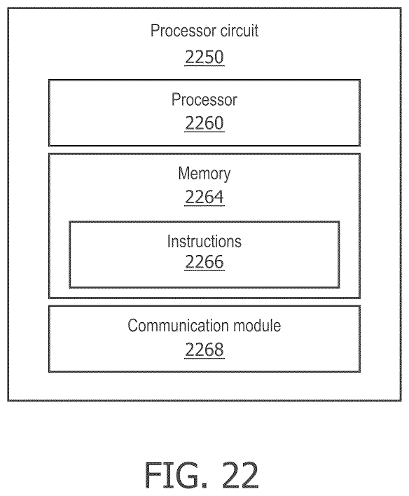

FIG. 22 is a schematic diagram of a processor circuit 2250, according to aspects of the present disclosure. The processor circuit 2250 may be implemented in the intravascular treatment system 100, the console 130, or other devices or workstations (e.g., third-party workstations, network routers, etc.), or on a cloud processor or other remote processing unit, as necessary to implement the method. As shown, the processor circuit 2250 may include a processor 2260, a memory 2264, and a communication module 2268. These elements may be in direct or indirect communication with each other, for example via one or more buses.

The processor 2260 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 2260 may also comprise another hardware device, a firmware device, or any combination thereof configured to perform the operations described herein. The processor 2260 may also be implemented as a combination of computing devices, e.g., a combination of a DSP and a microprocessor, a plurality of microprocessors, one or more microprocessors in conjunction with a DSP core, or any other such configuration.

The memory 2264 may include a cache memory (e.g., a cache memory of the processor 2260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an aspect, the memory 2264 includes a non-transitory computer-readable medium. The memory 2264 may store instructions 2266. The instructions 2266 may include instructions that, when executed by the processor 2260, cause the processor 2260 to perform the operations described herein. Instructions 2266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 2268 can include any electronic circuitry and/or logic circuitry to facilitate direct or indirect communication of data between the processor circuit 2250, and other processors or devices. In that regard, the communication module 2268 can be an input/output (I/O) device. In some instances, the communication module 2268 facilitates direct or indirect communication between various elements of the processor circuit 2250 and/or the system 100. The communication module 2268 may communicate within the processor circuit 2250 through numerous methods or protocols. Serial communication protocols may include but are not limited to United States Serial Protocol Interface (US SPI), Inter-Integrated Circuit (I²C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, preset sharing between the processor and central server, or readings from the pressure gauge) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

As will be readily appreciated by those having ordinary skill in the art after becoming familiar with the teachings herein, the combined laser atherectomy and pressure wave device advantageously provides a capability to both perform laser atherectomy and also fracture calcified vascular stenoses (including those in peripheral and coronary veins and arteries), without requiring a laser catheter to be moved relative to its surrounding sheath, and without introducing photoreactive materials, bubbles, or other reaction products (e.g., carbon monoxide, carbon dioxide, methane, water vapor, etc.) into the patient's bloodstream. The combined laser atherectomy and pressure wave device can be used in the field of cardiovascular procedures to treat chronic total occlusions (CTOs) with moderate to severe calcified lesions.

A number of variations are possible on the examples and aspects described above. For example, the combined laser atherectomy and pressure wave device could be used not only in arteries, but also in veins and other body lumens, including without limitation those of the liver, kidneys, stomach, gall bladder, lymphatic system, or otherwise. The system may employ other types of lenses, light sources, or fluids than those described herein, without departing from the spirit of the present disclosure. The combined laser atherectomy and pressure wave device could be used in other body lumens where there is clinical benefit in a shockwave, pressure wave, or vibration in combination with laser atherectomy. The combined laser atherectomy and pressure wave device can be detected from the design of the laser catheter and sheath design. A beam of light may contain multiple wavelengths, or may contain different polarities of the same wavelength of light, which act differently with the fluid(s) and/or with the optics, and that the laser catheter assembly may transmit light via liquid light guides instead of or in addition to optical fibers. In some aspects, the guidewire lumen may be adjacent to the optical assembly such that it does not cross the exit lens. For example, the guidewire may enter the tip between the exit lens and outer sheath and then travel through one of the fluid-conducting channels in the catheter. Such aspects fall within the scope of the present disclosure.

Accordingly, the logical operations making up the aspects of the technology described herein are referred to variously as operations, steps, objects, elements, components, or modules. Furthermore, it should be understood that these may occur, or be performed or arranged, in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the combined laser atherectomy and pressure wave device. Connection references, e.g., attached, coupled, connected, joined, or “in communication with” are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the combined laser atherectomy and pressure wave device as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter. Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. An intraluminal device for delivering laser light and pressure waves, the device comprising:
a flexible elongate member configured for positioning within a body lumen and comprising:
an outer sheath;
a laser catheter disposed within the sheath and fixedly coupled to the outer sheath;
a sealed lens disposed at a distal end of the outer sheath; and
a fluid chamber disposed within the sheath, distal of a distal end of the laser catheter and proximal of the sealed lens, wherein the fluid chamber is configured to be filled with a fluid;
wherein the outer sheath comprises a sealed acoustic window disposed radially outward from the fluid chamber,
wherein a first beam of laser light emitted by the laser catheter is configured to cause the fluid to generate a pressure wave,
wherein a second beam of laser light emitted by the laser catheter is configured to be transmitted via the fluid and the sealed lens,
wherein the sealed acoustic window and sealed lens prevent the fluid from entering the body lumen.

2. The intraluminal device of claim 1, wherein the flexible elongate member further comprises:
an inflow channel disposed within the outer sheath and in fluid communication with the fluid chamber; and
an outflow channel disposed within the outer sheath and in fluid communication with the fluid chamber.

3. The intraluminal device of claim 2,
wherein the fluid comprises at least one of a photoreactive fluid or an optically transmissive fluid,
wherein the fluid chamber is configured to be filled, via the inflow channel, with the photoreactive fluid and with the optically transmissive fluid at different times, and
wherein the fluid chamber is configured to be emptied, via the outflow channel, of the photoreactive fluid and of the optically transmissive fluid at different times.

4. The intraluminal device of claim 3, wherein the optically transmissive fluid comprises at least one of water, saline, or air.

5. The intraluminal device of claim 3, wherein the photoreactive fluid comprises an X-ray contrast fluid.

6. The intraluminal device of claim 1, wherein the sealed acoustic window is arranged relative to the fluid chamber such that the pressure wave is transmitted radially outward from the outer sheath into the body lumen.

7. The intraluminal device of claim 1, wherein the sealed lens is arranged relative to the fluid chamber such that the second beam of laser light is transmitted, into the body lumen, in a direction distal of the distal end of the sheath.

8. The intraluminal device of claim 2,
wherein the first beam of laser light comprises a first portion of a same beam of laser light emitted by the laser catheter and the second beam of laser light comprises a second portion of the same beam of laser light,
wherein the first portion of the same beam of laser light is transmitted via the fluid and the sealed lens simultaneously as the second portion of the same beam of laser light causes the fluid to generate the pressure wave.

9. The intraluminal device of claim 8,
wherein the flexible elongate member further comprises a beam splitter disposed within the outer sheath,
wherein the beam splitter directs the first portion of the same beam in a first direction and directs the second portion of the same beam in a different, second direction.

10. The intraluminal device of claim 2,
wherein the first beam of laser light comprises a first wavelength,
wherein the second beam of laser light comprises a different, second wavelength.

11. The intraluminal device of claim 1, wherein the laser catheter comprises an ultraviolet laser catheter.

12. The intraluminal device of claim 1, wherein the sealed lens comprises a flat proximal surface and a flat distal surface.

13. The intraluminal device of claim 1, wherein the sealed lens comprises a convex proximal surface.

14. The intraluminal device of claim 1, wherein the sealed lens comprises a convex distal surface.

15. The intraluminal device of claim 1,
wherein the flexible elongate member comprises a relay lens assembly,
wherein the relay lens assembly comprises the sealed lens.

16. The intraluminal device of claim 1, wherein the flexible elongate member further comprises a radiopaque marker positioned on the outer sheath proximate to the acoustic window.

17. The intraluminal device of claim 1, further comprising a guidewire lumen extending longitudinally through the laser catheter and the sealed lens.

18. An intravascular treatment system comprising:
a laser light source;
a photoreactive fluid source comprising a photoreactive fluid;
an optically transmissive fluid source comprising an optically transmissive fluid; and
an intravascular device configured to deliver laser light and pressure waves into a blood vessel, wherein the intravascular device comprises:
a flexible elongate member configured for positioning within the blood vessel and comprising:
an outer sheath;
a laser catheter disposed within the sheath and fixedly coupled to the outer sheath;
a sealed lens disposed at a distal end of the outer sheath; and
a fluid chamber disposed within the sheath, distal of a distal end of the laser catheter and proximal of the sealed lens;
wherein the outer sheath comprises a sealed acoustic window disposed radially outward from the fluid chamber,
wherein, when the fluid chamber is filled with the photoreactive fluid, a first beam of laser light emitted by the laser catheter causes the photoreactive fluid to generate a pressure wave to perform lithotripsy of a blood vessel blockage,
wherein, when the fluid chamber is filled with the optically transmissive fluid, a second beam of laser light emitted by the laser catheter is transmitted via the optically transmissive fluid and the sealed lens to perform laser atherectomy of the blood vessel blockage,
wherein the sealed acoustic window and sealed lens prevent the photoreactive fluid and the optically transmissive fluid from entering the blood vessel.

19. A laser atherectomy and pressure wave delivery method, comprising:
with a processor comprising a memory:
activating a pump;
via the pump and at least one inflow channel disposed within a flexible elongate member positioned within a blood vessel, controlling filling of a fluid chamber disposed within the flexible elongate member with an optically transmissive fluid;
activating a laser catheter disposed within the flexible elongate member, such that a first beam emitted by the laser catheter passes through the optically transmissive fluid and a sealed lens for laser atherectomy of a blood vessel blockage;
via the pump and a second inflow channel disposed within the flexible elongate member, controlling the filling of the fluid chamber with a photoreactive fluid; and
activating the laser catheter, such that a second beam emitted by the laser catheter passes through the fluid chamber, causing the photoreactive fluid to produce a pressure wave for lithotripsy of the blood vessel blockage,
wherein the sealed lens prevents the optically transmissive fluid and the photoreactive fluid from entering the blood vessel.
